(19) United States
(12) Patent Application Publication
(10) Pub. No.: US 2024/0010685 A1
(43) Pub. Date: Jan. 11, 2024

(54) RNA SENSORS AND USES THEREOF

(71) Applicants: YISSUM RESEARCH DEVELOPMENT COMPANY OF THE HEBREW UNIVERSITY OF JERUSALEM LTD, Jerusalem (IL); UNITED STATES OF AMERICA, AS REPRESENTED BY THE SECRETARY, DEPARTMENT OF HEALTH AND HUMAN SERVICES, Rockville, MD (US)

(72) Inventors: Eylon YAVIN, Jerusalem (IL); Daniel APPELLA, Rockville, MD (US)

(21) Appl. No.: 18/252,086

(22) PCT Filed: Nov. 8, 2021
(86) PCT No.: PCT/IL2021/051318

§ 371 Date: May 8, 2023

Related U.S. Application Data
(60) Provisional application No. 63/111,370, filed on Nov. 9, 2020.

(51) Int. Cl. C07K 14/00; A61K 49/00; C12Q 1/6816

(52) U.S. Cl. CPC C07K 14/003; A61K 49/0032; C12Q 1/6816

(57) ABSTRACT

The invention generally concerns a novel class of cyclopentane modified FIT-PNA (cpFIT-PNA) probes and uses thereof.

### TECHNOLOGICAL FIELD

The invention generally concerns a novel class of cyclopentane modified FIT-PNA (cpFIT-PNA) probes and uses thereof.

### BACKGROUND OF THE INVENTION

Sensitive and sequence-specific detection of RNA holds great promise for sensing RNA biomarkers in a variety of pathologies. It is clear that many types of RNA molecules other than mRNA (e.g., miRNA, IncRNA, and snRNA) may provide critical information on disease types and states. For example, the existence of certain point mutations in RNA (e.g. G12D KRAS and V600E BRAF SNPs for pancreatic and skin cancers, respectively) provides crucial information for optimal selection of drugs for use in personalized medicine.

Forced-intercalation-peptide nucleic acid (FIT-PNA) is a highly sensitive RNA/DNA sensor developed by the Seitz group in the early 2000's [1]. FIT-PNAs are “light-up” probes that are based on the replacement of one of the PNA monomers with a surrogate base. The surrogate base is a monomethine cyanine dye (e.g., Thiazole Orange (TO)) that contains a flexible methine bond. Upon FIT-PNA hybridization to complementary RNA, TO adopts a planar conformation and therefore becomes strongly fluorescent. It has been shown that FIT-PNAs may be tailored for sensitive detection of SNVs, dsRNA, viral RNA and IncRNAs.

Recently, red-shifted base surrogates for FIT-PNA (BisQ) [2] and FIT-DNA/LNA (QB) [3] have been reported. BisQ FIT-PNAs has been shown to detect oncogenic RNA in fresh human cancer tissues by simply spraying the FIT-PNA directly on the tissue. These finding highlight the diagnostic potential of such RNA sensing probes. To increase the brightness (where BR=λmax*QY) of FIT-DNA/LNA, a flanking rigid LNA base was introduced at the 3′-side of the surrogate base (TO or QB). By doing so, an increase of 4-5-fold in brightness [4] was achieved.

Cyclopentane modified PNAs are PNA monomers with a cyclopentane backbone (cpPNA). These cpPNAs have been shown to be outstanding DNA/RNA binders with single substitutions resulting in a dramatic increase in Tm's of 5-10° C. [5-8].

### BACKGROUND PUBLICATIONS

[1] O. Koehler, D. V. Jarikote and O. Seitz, ChemBioChem, 2005, 6, 69-77.
[2] N. Kolevzon, D. Hashoul, S. Naik, A. Rubinstein and E. Yavin, Chemical Communications, 2016, 52, 2405-2407.
[3] F. Hoevelmann, I. Gaspar, J. Chamiolo, M. Kasper, J. Steffen, A. Ephrussi and O. Seitz, Chemical Science, 2016, 7, 128-135.
[4] F. Hoevelmann, I. Gaspar, S. Loibl, E. A. Ermilov, B. Roeder, J. Wengel, A. Ephrussi and O. Seitz, Angewandte Chemie-International Edition, 2014, 53, 11370-11375.
[5] C. M. Micklitsch, B. Y. Oquare, C. Zhao and D. H. Appella, Analytical Chemistry, 2013, 85, 251-257.
[6] J. K. Pokorski, J. M. Nam, R. A. Vega, C. A. Mirkin and D. H. Appella, Chemical Communications, 2005, 2101-2103.
[7] J. K. Pokorski, M. A. Witschi, B. L. Purnell and D. H. Appella, Journal of the American Chemical Society, 2004, 126, 15067-15073.
[8] H. Zheng, M. Saha and D. H. Appella, Organic Letters, 2018, 20, 7637-7640.

### GENERAL DESCRIPTION

The inventors of the technology disclosed herein have developed a novel family of Forced-Intercalation-Peptide Nucleic Acid molecules (FIT-PNA), which exhibit increased brightness and greater sequence-specificity as compared to similar systems known in the art. According to the methodology disclosed herein, substituting neighboring bases to a surrogate base dye (such as BisQ) with cyclopentane modified PNA units (cpPNAs) affects the close environment of the probe, resulting in brighter and more sequence-specific FIT-PNAs. While the probe exemplified herein, for the sake of brevity, is BisQ, any monomethine dye may be similarly used. Non-limiting examples of other monomethine dyes are provided herein below.

The structure of BisQ and of a BisQ-PNA unit, wherein BisQ is a surrogate base in the PNA backbone, are shown below.

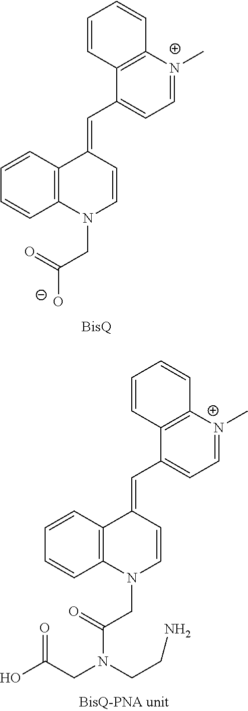 [SMILES: C[n+]1ccc(/C=C2\C=CN(CC(=O)[O-])c3ccccc32)c2ccccc21 ; C[n+]1ccc(/C=C2\C=CN(CC(=O)N(CCN)CC(=O)O)c3ccccc32)c2ccccc21]

For the sake of clarity, it should be noted that the conjugated system in both BisQ and BisQ-PNA unit, or in any of the other dyes mentioned herein, may be presented in different bond arrangements and the positive charge represented on one ring nitrogen atom may be similarly represented on the other ring nitrogen atom, depending on the bond arrangement used. The alternative structures are equivalent.

In a modified PNA molecule of the invention, herein a “conjugate”, a cyclopentyl (cp) moiety is provided at a base neighboring the position of the dye, e.g., BisQ, along the PNA backbone. The position of the cp unit(s) may be at any of positions A, B and/or C shown in Scheme 1 for a cpBisQ-PNA:

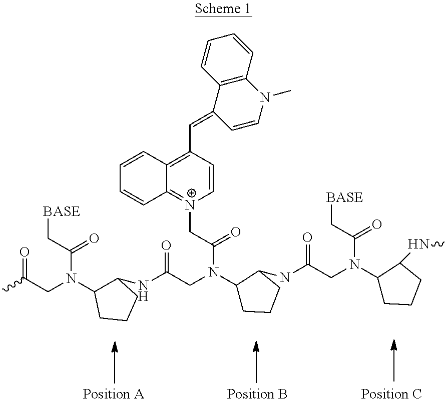

The “BASE” indicated in Scheme 1 is any nucleobase (or “base” for short) of the PNA.

Thus, in a first aspect of the invention, there is provided a peptide nucleic acid (PNA) comprising a PNA backbone and plurality of pendent bases, e.g., purine and pyrimidine bases, at least one of said bases is a surrogate base, wherein one or more bonds of the PNA backbone being a bond shared with a cyclopentyl group, and wherein the surrogate base is a monomethine dye.

Also provided is a cyclopentyl-modified PNA comprising a PNA backbone and a plurality of pendant bases, at least one of said pendent bases is a surrogate base and wherein one or more cyclopentyl groups are provided in proximity to said surrogate base, wherein the surrogate base is a monomethine dye.

Similarly provided is a cyclopentyl-modified PNA comprising a PNA backbone and a plurality of pendant bases, at least one of said pendent bases is a surrogate base selected amongst monomethine dyes and wherein one or more cyclopentyl groups are provided in proximity to said surrogate base.

Further provided is a cyclopentyl-modified forced-intercalation-peptide nucleic acid molecule (FIT-PNA) comprising a PNA backbone and a plurality of pendant bases, at least one of said pendent bases is a surrogate base selected amongst monomethine dyes and wherein one or more cyclopentyl groups are provided in proximity to said surrogate base.

In all aspects of the invention, the monomethine dye is not thiazol orange (TO).

Peptide Nucleic Acid (PNA) is a synthetic DNA analog that is uncharged and achiral. These properties provide PNA with excellent binding to complementary DNA and RNA. In addition, PNA is not recognized and degraded by proteases, peptidases or nucleases. Therefore, it is very stable in biological fluids and may be used as a diagnostic tool in-vivo and ex-vivo. Within the context of the invention, the PNA sequence has been modified to provide Forced Intercalation (FIT)-PNA molecules or conjugates in which a dye (e.g., BisQ) was introduced as a surrogate base, replacing one of the existing nucleotides in the PNA sequence. The cp unit is provided in vicinity or in proximity to the surrogate base. In other words, the FIT-PNA comprises a nucleobase or a combination of such bases which can intercalate or interact or associate or hybridize to a target RNA or DNA, a surrogate base, a cp unit and optionally other modified bases as disclosed herein.

The FIT-PNA comprises a complementary region that may be any base sequence that is complementary with a target nucleic acid sequence in a cell or tissue, as defined herein. The target sequence may be any sequence capable of interacting or hybridizing with a sequence of interest (SOI) present in a sample and which presence is indicative of a mutation, a condition or a disease, or presence of a particular organism. For example, the target sequence may be indicative of the presence of an organism, such as a parasite, in a host subject being administered with a FIT-PNA of the invention. Alternatively, the target sequence may be indicative of an acquired genetic resistance of an organism to a substance, e.g., a drug.

The “surrogate base” refers to one or more dyes which replace one or more (typically one or two) existing PNA nucleobases. The presence of the surrogate base does not alter the PNA backbone. The surrogate base is typically a dye having a single methine bond (thus being a monomethine). The dye is selected to exhibit quenched fluorescence when the dye is associated to the PNA and to exhibit fluorescence emission when the PNA is hybridized to a target sequence. The dye is a red-to-NIR emitting dye, which is not (or which is different from) thiazol orange (TO). In other words, the dye is a material capable of switching on fluorescent emission at wavelengths between about 600 nm to 800 nm, within the red-to-NIR or far-red radiation spectrum, upon inducing a change in the dye structural conformation, in connectivity or association to a complementary component, or upon induing any change in the dye's steric degrees of freedom.

In some embodiments, the monomethine dye is a cyanine dye.

The cyanine dyes may be BisQ or Dye 1 or Dye 2:

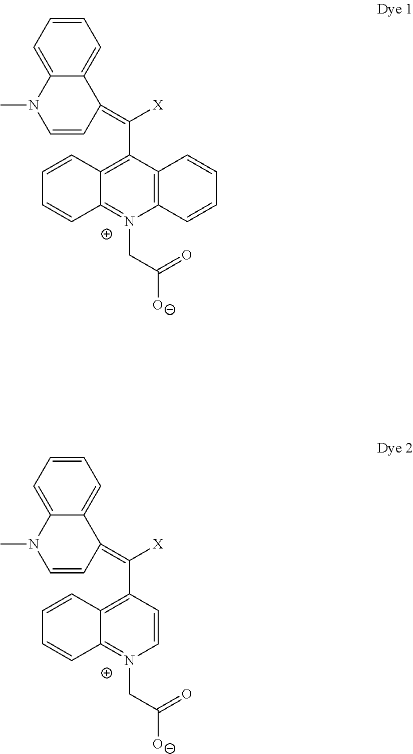

Dye 1 and Dye 2 are shown below when provided on a PNA or cpPNA backbone, wherein each X represents a halogen atom (e.g., F, Cl, Br or I):

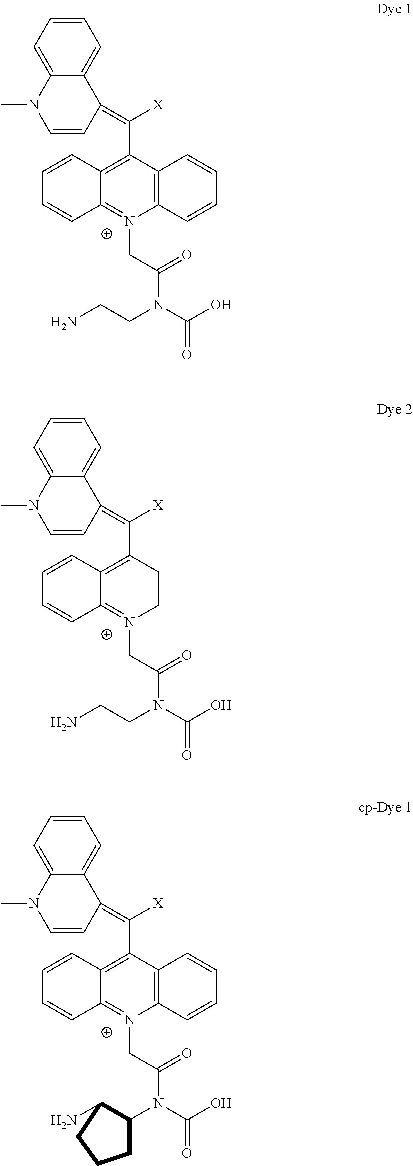

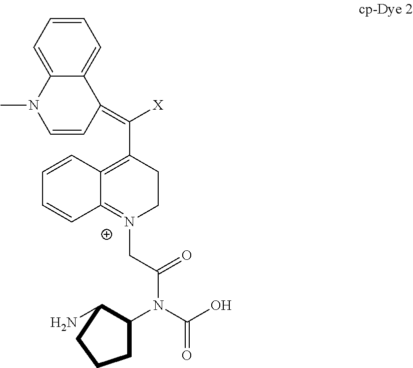

The position of the surrogate base is governed by the length of the FIT-PNA sequence and is generally at or in proximity to the center of the sequence. For example, for a 12-mer sequence the surrogate base would be anywhere between base 4 to base 9.

As indicated herein, one or more cyclopentyl (cp) groups are positioned in proximity or in the vicinity of the surrogate base. The distance from the surrogate base is typically not greater than one base. In some embodiments, the cp group is positioned on the PNA backbone between the position of the surrogate base and the position of the next nucleotide base (as in Position A in Scheme 1), across a nucleotide base (as shown in position C in Scheme 1) or on the FIT-PNA unit carrying the surrogate base (as in Position B in Scheme 1). In some embodiments, the cp group is on the 3′ end of the FIT-PNA or on the 5′ end of the FIT-PNA.

In some embodiments, for each surrogate base, the FIT-PNA comprises 1 or 2 or 3 cp groups. In some embodiments, the number of cp groups per surrogate group is 1 or 2. In some embodiments, the FIT-PNA comprising 1 or 2 surrogate bases and 1 or 2 cp groups.

In some embodiments, the cp group is 1 to 7 atoms away from the FIT-PNA amide nitrogen atom substituting the surrogate base. The numbering of atoms is shown in Scheme 2 below. The depicted BisQ may be any of the other dyes mentioned herein.

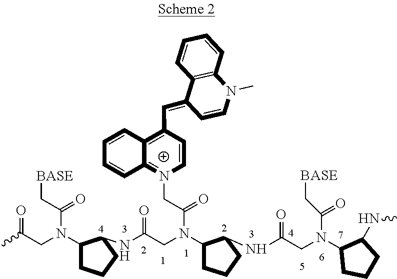

In some embodiments, each conjugate comprises 1 surrogate base and 1 cp group.

The FIT-PNA used in accordance with products and methods of the invention may be of any length. Typically, the FIT-PNA comprises between 8 and 20 base units. In some embodiments, the number of bases in a PNA is between 4 and 30, 4 and 25, 4 and 20, 4 and 15, 4 and 10, 8 and 30, 8 and 25, 8 and 20, 8 and 15, 8 and 10, 10 and 30, 10 and 25, 10 and 20, 10 and 15, 15 and 30, 15 and 25 or between 15 and 20 bases. In some embodiments, the number of bases is between 4 and 20 or 4 and 15. In some embodiments, the number of bases is 4, 5, 6, 7, 8, 9, 10, 11, 12, 13, 14, 15, 16, 17, 18, 19 or 20. In some embodiments, the number of bases is 11.

In some embodiments, the cp is positioned between the surrogate base and the 5′ end of a FIT-PNA having between 8 and 20 bases. In some embodiments, the cp is positioned between the surrogate base and the 3′ end of a FIT-PNA having between 8 and 20 bases.

In some embodiments, the cp is positioned at the 3′ end of the FIT-PNA.

In some embodiments, the cp is positioned at the 5′ end of the FIT-PNA.

As shown in schemes and figures herein, the cp group is positioned along the FIT-PNA backbone such that one of the cylopentyl bonds is a bond of the PNA backbone. In other words, one of the cyclopentyl bonds is shared with the backbone. The cyclopentyl group is not pendent from the probe unit or the PNA backbone.

Conjugates of the invention, refer to as cyclopentyl-modified or cpPNA molecules having one or more surrogate bases, are configured as forced-intercalation-peptide nucleic acid molecules (FIT-PNA) which can be used as PNA-based probes in DNA/RNA-based diagnostic methods. In these cpFIT-PNA-based conjugates, the flexibility of the methine bond of the dye results in a nonplanar conformation leading to quenching of the dye fluorescence. When hybridized into DNA or RNA, the dye is forced to adopt a planar conformation resulting in significant enhancement of fluorescence. The increase in fluorescence is highly sensitive to local structural perturbations induced by an adjacent base mismatch permitting detection of single nucleotide mismatch in DNA or RNA.

To exemplify the ability to increase brightness and sequence specificity, the inventors have synthesized various FIT-PNAs with flanking cpPNAs. Some non-limiting examples include:
3′-dK₄-ATA-C-BisQ-cpT-ACAAC-5′
3′-dK₄-ATA-cpT-BisQ-T-ACAAC-5′
3′-dK₄-ATA-cpC-BisQ-cpT-ACAAC-5′
3′-dK₄-ATACA-cpT-BisQ-C-AAC-5′
3′-dK₄-ATACA-cpT-BisQ-cpC-AAC-5′.

As used in the sequences, dK₄ is a short cell penetrating peptide (CPP) comprised e.g., of 4 D-Lysine groups and the position of the cp is either left or right to BisQ. For example, in the first sequence, 3′-dK₄-ATA-C-BisQ-cpT-ACAAC-5′, the cp modification is on the T base adjacent to the BisQ from the 5′-end.

Table 1 lists several 11-mers prepared and tested. In the first set of FIT-PNAs (cpFIT-PNAs 2-4), both mono and bis cpPNA substitutions (FIT-PNA without cpPNA served as a control, FIT-PNA 1) were introduced. In the 2nd set, the location of BisQ was shifted and 2 cpPNAs (cpFIT-PNAs 6 and 7 where FIT-PNA 5 served as a control) units were introduced. FIG. 1A and FIG. 1B present fluorescence measurements of cpFIT-PNAs with complementary RNA.

TABLE 1
11-mer FIT-PNAs according to the invention. BisQ
appear in bold and cyclopentane modified PNA
bases are in italics. 1 and 5 FIT-PNAs serve as
a control for each set.

| | PNA No. | Sequence |
|---|---|---|
| Set 1 | 1 | ^{3′}(4)K_{D}-ATA-C-BisQ-TACAAC-^{5′} |
| | 2 | ^{3′}(4)K_{D}-ATA-C-BisQ-cpT-ACAAC-5′ |
| | 3 | ^{3′}(4)K_{D}-ATA-cpT-BisQ-T-ACAAC-^{5′} |
| | 4 | ^{3′}(4)K_{D}-ATA-cpC-BisQ-cpT-ACAAC-5′ |
| Set 2 | 5 | ^{3′}(4)K_{D}-ATACA-T-BisQ-C-AAC-^{5′} |
| | 6 | ^{3′}(4)K_{D}-ATACA-cpT-BisQ-C-AAC-^{5′} |
| | 7 | ^{3′}(4)K_{D}-ATACA-cpT-BisQ-cpC-AAC-^{5′} |

In both series of PNAs shown in Table 1, a remarkable increase in FIT-PNA fluorescence was observed when both flanking bases were substituted with cpPNA (FIT-PNAs 4 and 7 for set 1 and 2, respectively). Compared to unmodified FIT-PNAs (1 and 5), a 2-3-fold increase in fluorescence was noted. Quantum yields (QY) for all FIT-PNA were determined using Cresyl Violet as a reference fluorescent dye. For double-cp modified FIT-PNA (4 and 7), a significantly higher QY was observed (0.33 and 0.39 for 4 and 7, respectively, Table 2). These values translate to a brightness that is unprecedented for FIT-PNAs (Table 2).

TABLE 2
photophysical properties of duplex
cpFIT-PNAs with complementary RNA.

| | | εₘₐₓ | | BR | | | |
|---|---|---|---|---|---|---|---|
| FIT- | λ_{max, abs} | [mM⁻¹ | | [mM⁻¹ | | | |
| PNA | (nm) | cm⁻¹] | Φ | cm⁻¹] | I/I₀ | Tₘ | ΔTm |
| 1 | 588 | 83.2 | 0.18 | 15.0 | 12 | 50.2 | |
| 2 | 590 | 91.0 | 0.18 | 16.4 | 15 | 53.7 | (+3.5) |
| 3 | 589 | 95.2 | 0.27 | 25.7 | 12 | 53.7 | (+3.5) |
| 4 | 590 | 85.8 | 0.33 | 28.3 | 46 | 54.7 | (+4.5) |
| 5 | 587 | 82.3 | 0.20 | 16.5 | 24 | 51.8 | |
| 6 | 586 | 85.7 | 0.34 | 29.1 | 46 | 53 | (+1.2) |
| 7 | 586 | 90.6 | 0.39 | 35.3 | 47 | 52.9 | (+1.1) |

In addition, the enhancement upon duplex formation with complementary RNA observed for these FIT-PNAs (4 and 7) was quite dramatic; 47-fold for FIT-PNA 7 and 46-fold for FIT-PNA 4 (FIG. 1B). The effect of cpPNA monomers to duplex stability was assessed by measuring Tm for all RNA:PNA duplexes (Table 2). The stabilizing effect was smaller for FIT-PNA in comparison to that reported for cpPNA. It is believed that this is due to the close proximity of the modified bases to the bulkier BisQ surrogate base in comparison to natural bases. For all measurements an additional transition at 60° C. was observed. Table 2 summarizes the photophysical properties of cpFIT-PNAs.

It is worth noting that when the cyclopentane ring is introduced at the carboxy side of BisQ (left to BisQ, see Table 1 and Scheme 1), the 5-membered ring is only 4 atoms away from the nitrogen atom on the backbone that is connected to the surrogate base (BisQ). When the cyclopentane is positioned on the other side of BisQ, a distance of 7 atoms results. Without wishing to be bound by theory, this difference in location seems to have a substantial effect of cpPNA on FIT-PNA fluorescence. Thus, cpPNA mono-substitutions at the carboxy side to BisQ (FIT-PNAs 3 and 6) results in comparably high QYs for a single substitution (0.27 and 0.34, respectively), whereas a single substitution at the amino side to BisQ (FIT-PNA 2) does not increase the QY (0.18) compared to unmodified FIT-PNA (FIT-PNA 1) and has a small effect on FIT-PNA brightness (16.4 for FIT-PNA 2 vs. 15 for FIT-PNA 1, Table 1).

Mismatch sensitivity was evaluated for single and doubly modified cpFIT-PNAs. Mismatch sensitivity for mono-substituted cpPNAs (cpFIT-PNAs 2, 3, and 6) are presented in FIGS. 3A-B. Double substituted cpFIT PNAs 4 and 7 were compared to FIT-PNA controls (1 and 5) and are presented in FIGS. 2A-B. 11-mer RNAs with all three possible mismatches were introduced either at 3′ (to the left of BisQ) or 5′ (to the right of BisQ) positions opposite to the nearby base to BisQ. One clear observation is that cpFIT-PNAs have significant selectivity for pyrimidine-pyrimidine mismatches (CC, TU, TC, CU where underlined base refers to PNA base) in comparison to FIT-PNA controls with the exception of the CU mismatch for cpFIT-PNA 4. For cpFIT-PNA 7, TC and TU mismatches are discriminated by a factor of 7.5 and 13.9-fold (vs. full matched RNA), respectively. This is around a 3 to 5-fold increase with respect to non-modified FIT-PNA 5.

As noted herein, the cp group may be provided at either or both sides of the surrogate base or directly on the surrogate base backbone, as shown in Scheme 1. As demonstrated herein, positioning the cp group 1 carbon away from the amide nitrogen (Scheme 2) increased the brightness and selectivity of FIT-PNA probes. The closer the cyclopentane is to the surrogate base, the higher performance of the FIT-PNA.

In another example, the following FIT-PNAs were synthesized for detecting the V660E BRAF point mutation (PNA sequence 5′ (NH₂)→3′ (COOH), also shown in Table 3:
GATTT-BisQ-TCTGTAGCTAC-CLIP6,
GATTT-BisQ-TCTGTAGCTAC-PEG₈-CLIP6,
GATTcpT-BisQ-TCTGTAGCTAC-PEG₈-CLIP6.

The 17-mer FIT-PNAs listed in Table 3 have a different CPP, namely, CLIP6 (where CLIP6=KVRVRVRV^{D}PPTRVRERVK).

TABLE 3
FIT-PNAs targeting the BRAF V600E point mutations. PNA sequences were
all conjugated to the CLIP6 peptide. Cp = cyclopentane PNA monomer.

| PNA sequence 5′(NH2)→3′(COOH) | Target Gene | Cell Lines |
|---|---|---|
| GATTT-BisQ-TCTGTAGCTAC-CLIP6 | BRAF, V600E | SKMe1_28/A2058/A375 and MeW [symbol] |
| GATTT-BisQ-TCTGTAGCTAC-PEG₈-CLIP6 | BRAF, V600E | SKMe1_28/A2058/A375 and MeW [symbol] |
| GATTcpT-BisQ-TCTGTAGCTAC-PEG₈-CLIP6 | BRAF, V600E | SKMe1_28/A2058/A375 and MeW [symbol] |

[symbol]

indicates data missing or illegible when filed

CLIP6 CPP was chosen as an alternative CPP to the one used in Table 1 (dK₄). This was based on the effective cellular uptake observed for this CPP when conjugated to a splice switching PNA. Cellular uptake into glioblastoma cancer cells (U87) occurs via a productive non-endosomal mechanism. However, CLIP6 was found to quench FIT-PNA fluorescence (FIG. 4). To overcome this hurdle, several FIT-PNAs (targeting the oncogenic IncRNA HOTAIR) have been prepared with a PEG linker at varying lengths in order to reduce the quenching effect (Table 4).

TABLE 4
HOTAIR FIT-PNA with CLIP CPP attached via PEG linkers.

| Name | Description | Construct (3′ to 5′) |
|---|---|---|
| CLIP-Hotair1 | PNA conjugated to CLIP | CLIP-3′TGTTCTGGTC-BisQ- |
| | | CTCGAC-5′ |
| CLIP-PEG3-Hotair1 | PNA conjugated to PEG3 linker and CLIP | CLIP-PEG3-3′-TGTTCTGGTC-Bi [symbol] |
| | | CTCGAC-5′ |
| CLIP-PEG8-Hotair1 | PNA conjugated to PEG8 linker and CLIP | CLIP-PEG8-3′-TGTTCTGGTC-Bi [symbol] |
| | | CTCGAC-5′ |
| CLIP-PEG12-Hotair1 | PNA conjugated to PEG12 linker and CLIP | CLIP-PEG12-3′-TGTTCTGGTC- |
| | | BisQ-CTCGAC-5′ |

[symbol]

indicates data missing or illegible when filed

As shown in FIG. 4, direct conjugation of CLIP to FIT-PNA caused a significant decrease in fluorescence in comparison to the duplex formed with the dK₄ peptide. The PEG8 linker restored most of the fluorescence for the CLIP6-PEG₈-HOTAIR FIT-PNA.

FIGS. 5A and 5B present the fluorescence spectra of the 2 FIT-PNAs that do not include cp with synthetic RNA (either with/out point mutations). The data points to the added value of introducing the PEG8 linker. Clearly, the addition of the PEG₈ linker improves the signal for fully matched RNA without losing the discrimination for the SNP. The addition of cpPNAs on both sides of BisQ (Table 2) results in an increased fluorescence in signal with mutant RNA (FIG. 6), however, the signal for WT RNA (with mismatch) also increases. This leads to some loss of discrimination for this SNP.

Methylating the N-7 position of Guanosine base of a PNA monomer (G+ PNA monomer) results in an improvement in the hybridization to complementary DNA whilst avoiding PNA:PNA inter- and intra-duplex formation. As enhancement of FIT-PNA fluorescence depends on the ratio between ss/ds forms (PNA/PNA:RNA), lowering the background fluorescence of ssPNA should result in greater sensitivity (i.e. increased fluorescence enhancement). In the ss form, the FIT-PNA has low background fluorescence due to 7E-7E interactions between the aromatic BisQ and adjacent aromatic PNA monomers (especially purines: G and A). As BisQ has a delocalized positive charge (from nitrogen to nitrogen), the introduction of an adjacent positive charge on G or A causes electrostatic repulsion between BisQ and G+/A+ resulting in reduced background fluorescence. As a proof of concept, the G+ monomer has been successfully introduced into the CCAT1 FIT-PNA sequence (CCAACCT-BisQ-G⁺TAAGTG-dK₄). Data points to both a decrease in signal background of ssFIT-PNA and to an increase in fluorescence in duplex form (FIT-PNA:RNA) in comparison to non-modified CCAT1 FIT-PNA (FIG. 7). It is not clear why the increase in signal occurs for duplex FIT-PNA:RNA.

Notwithstanding the reason, the increase adds to a better performance of the FIT-PNA that is modified with G+ and highlights the potential of combining G+/A+ with cpG/cpA in the same PNA monomer.

Thus, in a conjugate of the invention comprising a dye, e.g., BisQ and a cp unit and multiple PNA bases selected from purine (guanine (G) and adenine (A)) and pyrimidine bases (cytosine (C) and thymine (T)), one or more of the guanine bases is a charged base, herein labeled G⁺ and/or one or more of the adenine bases is a charged base, herein labeled A⁺. The charged bases are alkylation products of the free nucleobases, wherein the charged bases may be depicted as having the structures shown in Scheme 4a below. In the structures shown, each group Z may be an alkyl group, typically but not necessarily selected amongst alkyl groups having between 1 and 5 carbon atoms. In some cases, as depicted in Scheme 4b, the alkyl group is a methyl group.

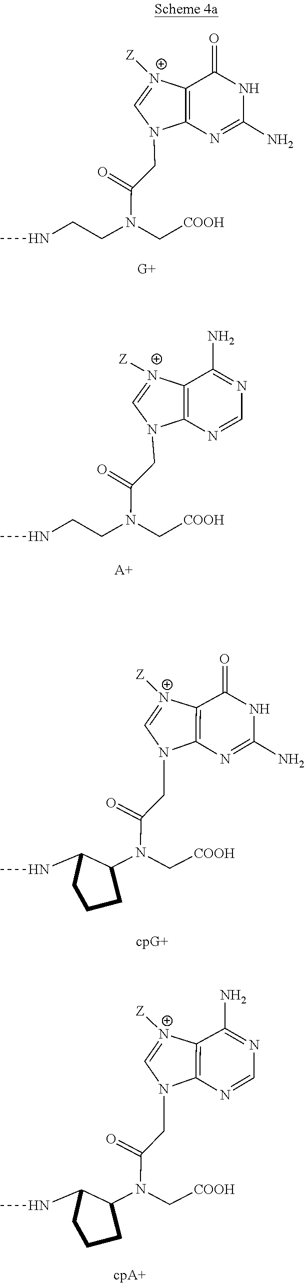

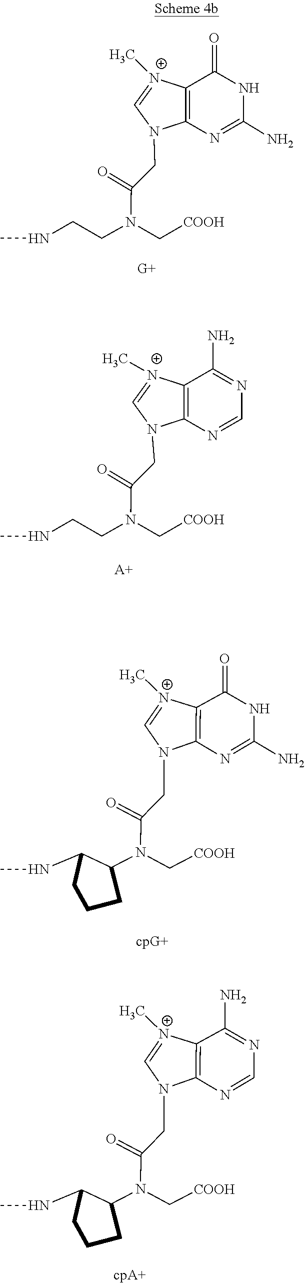

In some embodiments, the modified or charged bases, G⁺ and A⁺, may be provided with a cp group positioned in their vicinity or as a cpG⁺or cpA⁺ monomer, in similarity to cpBisQ (Scheme 4). The cpG⁺ monomer has been successfully introduced into the CCAT1 FIT-PNA sequence (CCAACCT-BisQ-cpG⁺TAAGTG-dK₄). Data points to an increase in fluorescence in duplex form (FIT-PNA:RNA) in comparison to non-modified CCAT1 FIT-PNA as well as G+ and cpG modified CCAT1 FIT-PNA sequences (FIG. 8).

Another approach for modulating FIT-PNAs photophysical properties is by introducing an oxetane ring on the PNA backbone. The modification was used to introduce the group at either/or both sides of BisQ or directly on the BisQ monomer. The structure of the oxetane unit on a PNA unit is shown in Scheme 5 below:

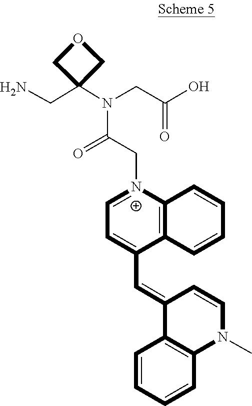

Thus, the invention further provides a cyclopentyl-modified FIT-PNA comprising a PNA backbone and a plurality of nucleobases, at least one of said bases is a surrogate base and wherein one or more cyclopentyl groups are provided in proximity to said surrogate base, wherein the surrogate base is a monomethine dye, e.g., a cyanine dye; and wherein the FIT-PNA optionally further comprises a charged guanine base (G+ or cpG+) and/or a charged adenine base (A+ or cpA+) (as shown in Scheme 4a or 4b) and further optionally an oxetane-modified surrogate base (as shown in Scheme 5). cp-Modified PNA conjugates of the invention are thus selected from:
(i) cp-modified FIT-PNA molecules comprising a probe unit, e.g., a BisQ unit;
(ii) cp-modified FIT-PNA molecules comprising a probe unit, e.g., a BisQ unit, and a G+ or A+ base;
(iii) cp-modified FIT-PNA molecule comprising a probe unit, e.g., a BisQ unit, and a cpG+ and cpA+ base;
(iv) cp-modified FIT-PNA molecule comprising a probe unit, e.g., a BisQ unit, and an oxetane-modified base;
(v) cp-modified FIT-PNA molecule comprising a probe unit, e.g., a BisQ unit, a G+ and/or A+ base, a cpG+ and cpA+ base, and an oxetane-modified base.

For their superior performances as detection probes, cpFIT-PNA conjugates of the invention may be used for the detection of RNA and/or DNA in a sample.

Thus, in most general terms, the invention further provides a method for detecting or for determining presence of a sequence of interest (SOT) in a sample, the method comprising contacting said sample with a cpFIT-PNA according to the invention under conditions permitting hybridization of said cpFIT-PNA with the SOI and detecting emission of light in the red-to-NIR region upon exposure to red-to-NIR radiation. Where emission is detected, the sample is said to contain an amount of the SOI, wherein emission is not detected, the sample is said not to contain an amount of the SOI, or is said to contain an amount of the SOI that is below the limit of detection.

As used herein, the term “amount” made in reference to presence of an SOI in a sample, refers to a quantity of the SOI that is sufficient to interact with a conjugate of the invention to provide a measurable signal. This amount is in the low nM range as shown for FIT-PNA 7 (Table 2).

Once a sample is exposed to the cpFIT-PNA, it may be incubated for a period of time sufficient to allow hybridization with the SOI. Incubation may proceed at room temperature (between 23 and 30° C.) for a period of between 5 and 180 minutes. Thereafter, the sample may be exposed to a red-to-NIR fluorescence detector for determining emission from said sample. In cases the sample is an in vivo sample, namely a tissue in the subject's body, the tissue is contacted with the cpFIT-PNA and imaged using a red—to NIR fluorescence detector.

The ability to determine ‘on demand’ or ‘on the spot’ presence of a SOI permits immediate determination of malignancies in fluorescence guided surgery by directly applying, e.g., by spraying, the cpFIT-PNA on an inspected tissue during cyto-reductive surgery. In such a procedure, use of a conjugate of the invention aims at determining the extent of removal of a malignant tissue during or after a malignancy removal surgery; the method employing the conjugate comprises exposing a tissue removed and incubated with a conjugate of the invention to a red-to-NIR fluorescence detector, to detect emission of red-to-NIR light from said tissue. If the borders of the removed tissue emit light, this will indicate the presence of a remaining malignant tissue in said subject, which can be further removed and evaluated until the sample emits no further light in response to red-to-NIR radiation.

The “sequence of interest (SOI)” is any RNA or DNA sequence which presence in a sample is to be qualitatively or quantitatively determined. The sequence may be any RNA/DNA sequence that is associated with a disease or a condition, any RNA/DNA indicative of a genetic condition, any pathogenic RNA/DNA, and others. In some embodiments, the SOT is an RNA such as an oncogenic RNA, e.g., mRNA, ncRNA, or miRNA; or pathogenic RNA, e.g., viral RNA, bacterial RNA, and parasite RNA. In some embodiments, the SOI is a DNA, wherein the conjugate is used to detect single point mutations associated with a genetic disorder.

In some embodiments, the SOI is RNA/DNA present in living cancer cells, viral-infected cells, or malaria-infected red blood cells.

In some embodiments, detection of the SOI is in the course of determining presence of a bacterium.

A “sample” may be any sample containing or in a form of cells or tissues presented and evaluated in vivo or ex vivo. The sample may be a blood sample, a plasma sample, a skin tissue, a mucosal tissue, a tissue sample suspended in a medium, or a tissue present in vivo. Where the cpFIT-PNA is used in vivo, it may be applied to the tissue by any means capable of delivering the conjugate to the tissue, optionally under conditions permitting internalization of the conjugate. Where cellular internalization may be required, a conjugate comprising at least one moiety designed for cellular internalization may be used. This moiety may, for example, may be an amino acid (peptide) sequence.

In some embodiments, the amino acid sequence comprises D-lysines (for example at the PNA's C-terminus) or PEG₈-CLIP6 (KVRVRVRVDPPTRVRERVK). In some embodiments, the at least one moiety designed for cellular internalization is a fatty acid moiety, such as a stearyl group.

The sample may be treated with the cpFIT-PNA of the invention by adding the conjugate to the sample, by adding a sample onto the conjugate, by mixing the conjugate with the sample, by spraying the sample with the conjugate, by incubating the sample with the conjugate or by any other means.

Where the sample is or comprises a tissue sample, the tissue may be excised from a subject and incubated with a conjugate of the invention.

Methods of the invention for detecting or determining presence of a sequence of interest (SOI) in a sample may be used for determining presence, development or progression of a disease or a condition; or a genetic condition; or presence of a pathogen or a parasite in a sample (or in a subject-being a human or an animal subject). Methods of the invention further provide means for determining viral, malaria or bacterial infections in a subject.

Thus, in another of its aspects, the invention provides a method for determining presence, development or progression of a disease or a condition; or a genetic condition; or presence of a pathogen or a parasite in a sample, the method comprising contacting a sample suspected of containing an SOI indicative of presence of a disease, condition or pathogen or parasite in a subject with a cpFIT-PNA according to the invention under conditions permitting hybridization of said cpFIT-PNA with the SOI and detecting emission of light in the red-to-NIR region upon exposure to red-to-NIR radiation. Where emission is detected, the sample is said to contain the SOI, wherein emission is not detected, the sample is said not to contain the SOI, or is said to contain an amount of the SOI that is below the limit of detection. Where presence of the SOI is determined, a determination may be made as to the presence, development or progression of the disease or the condition; or the genetic condition; or the presence of the pathogen or the parasite in the sample or in the subject from which the sample is obtained.

In some embodiments, the disease or condition is a cancerous condition or disease that is manifested in abnormal cell growth capable of invading adjacent tissues, and optionally further capable of spreading to distant tissues. Such include adrenocortical carcinoma, bladder cancer, bone cancer, osteosarcoma, malignant fibrous histiocytoma, breast cancer, burkitt lymphoma, carcinoid tumor, cerebellar astrocytoma, cerebral astrocytoma/malignant glioma, cervical cancer, colon cancer, cutaneous t-cell lymphoma, desmoplastic small round cell tumor, endometrial cancer, ependymoma, oesophageal cancer, Ewing's sarcoma, extragonadal germ cell tumor, extrahepatic bile duct cancer, eye cancer, retinoblastoma, gallbladder cancer, head and neck cancer, heart cancer, hepatocellular cancer, Hodgkin lymphoma, hypopharyngeal cancer, intraocular melanoma, islet cell carcinoma, Kaposi sarcoma, laryngeal cancer, liver cancer, lung cancer, lymphomas, medulloblastoma, melanoma, merkel cell carcinoma, mesothelioma, mouth cancer, mycosis fungoides, nasopharyngeal carcinoma, neuroblastoma, non-small cell lung cancer, oropharyngeal cancer, ovarian cancer pancreatic cancer, parathyroid cancer, penile cancer, pharyngeal cancer, pleuropulmonary blastoma, prostate cancer, rectal cancer, renal cell carcinoma, retinoblastoma, rhabdomyosarcoma, salivary gland cancer, stomach cancer, testicular cancer, throat cancer, thymic carcinoma, thyroid cancer, urethral cancer, uterine cancer, endometrial, uterine sarcoma, Wilms tumor.

In some embodiments, the malignancy is cancer, e.g., ovarian cancer or any of the cancers known in the art.

The genetic condition to be determined by processes of the invention is any condition, disorder or disease that is caused by one or more abnormalities in the genome of a subject. The genetic condition, disorder or disease may be congenital, hereditary or caused by new mutations or changes to the DNA of the subject. In some embodiments, said genetic condition, disorder or disease is a single gene mutation (autosomal dominant, autosomal recessive, X-linked dominant, X-linked recessive, Y-linked, mitochondrial). In some embodiments, the genetic condition, disorder or disease is polygenic. Multifactorial disorders, conditions and disease include, but are not limited to heart disease, diabetes, asthma, autoimmune diseases such as multiple sclerosis, cancers, ciliopathies, cleft palate, hypertension, inflammatory bowel disease, intellectual disability, mood disorder, obesity, refractive error, infertility and so forth. None limiting examples of such diseases and disorders include DiGeorge syndrome, Angelman syndrome, Canavan disease, Charcot-Marie-Tooth disease, Cri du chat, Cystic fibrosis, Down syndrome, Duchenne muscular dystrophy, Haemophilia, Klinefelter syndrome, Neurofibromatosis, Phenylketonuria, Prader-Willi syndrome, Sickle-cell disease, Tay-Sachs disease and Turner syndrome.

In some embodiments, the genetic condition, disorder or disease is associated with single nucleotide polymorphism (SNP).

Detection of the SOI may be sample by sample or by multiplexing, namely by simultaneous detection of different sequences (SOIs) using a single conjugate of the invention, or by simultaneous detection of a single SOI using a plurality of different conjugates. High-throughput multiplexing, array-based platforms are the most popular techniques in clinical diagnostics. High-density arrays of microspots (down to picoliter volumes) are easily implemented and analyzed for diagnostic purposes, wherein the signal readout is mainly achieved by optical methods. Optical microarray systems often rely on fluorescence detection allow automatic assay preparation. The intensities of the bound fluorescently labeled conjugates or hybridized products may be measured via laser scanning or observed with a scanning charge-coupled device (CCD) or any other device operable in the red-to-NIR regime.

Thus, in some embodiments, conjugates of the invention are provided in an array-based system comprising a plurality of detection regions, each region containing or is associated with a different sample and configured to receive a single conjugate (FIT-PNA) of the invention; or each region contains or is associated with different conjugates and configured to receive a single sample. This arrayed system provides a rapid and high-throughput diagnostics.

The array may be provided in a variety of forms and shapes. In some embodiments, conjugates of the invention may be provided on a solid support, namely attached or associated to a solid surface through either covalent or non-covalent bonds. Such solid surfaces may be aero gels, hydro gels, resins, beads, biochips, micro fluidic chip, a silicon chip, multi-well plates such as microtiter plates or microplates, membranes, filters, conducting and non-conducting metals, glass and magnetic supports. More specific examples of useful solid supports include silica gels, polymeric membranes, particles, derivative plastic films, glass beads, cotton, plastic beads, alumina gels, polysaccharides such as Sepharose, nylon, latex bead, magnetic bead, paramagnetic bead, super paramagnetic bead, starch and the like. This also includes, but is not limited to, microsphere particles, magnetic beads, charged paper, functionalized glass, germanium, silicon, PTFE, polystyrene, gallium arsenide, gold, and silver.

The invention also provides sensors, e.g., RNA sensors, in a form of cpFIT-PNA of the invention.

The invention further provides a kit comprising a conjugate according to the invention and instructions of use.

In some embodiments, the kit further comprises a medium for mixing therein a sample and the conjugate (FIT-PNA).

In some embodiments, the kit comprises the conjugate of the invention provided on a solid support, as defined herein.

In some embodiments, the conjugate is provided in a medium.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-C demonstrates how cpPNA enhances fluorescence in all FIT-PNAs. (A) set 1 showing fluorescence of FIT-PNA:RNA duplex for FIT-PNAs 1-4, (B) set 2 showing fluorescence of FIT-PNA:RNA duplex for FIT-PNAs 5-7 and, (C) enhanced fluorescence of bis-cpFIT-PNAs (4 and 7) after RNA hybridization. [FIT-PNA]=[cpFIT-PNA]=3 μM, [RNA]=4.5 μM.

FIGS. 2A-B demonstrate mismatch sensitivity of double substituted cpFIT-PNAs compared to FIT-PNA controls. (A) double cpFIT-PNA 4 compared to FIT-PNA 1 and (B) double cpFIT-PNA 7 compared to FIT-PNA 5. FIT-PNAs were annealed to all possible 11-mer RNAs and measured on a plate reader (λex=575 λnm, Em=619 nm, n=3). [FIT-PNA]=[cpFIT-PNA]=0.5 μM, [RNA]=0.75 μM.

FIGS. 3A-B provide mismatch sensitivity of single substituted cpFIT-PNAs. (A) cpFIT-PNA 2 and 3 and (B) cpFIT-PNA 6. FIT-PNAs (0.5 μM) were annealed to all possible 11-mer RNAs (0.75 μM) and measured on a plate reader (λex=575 nm, λem=619 nm, n=3).

FIG. 4 provides HOTAIR FIT-PNA fluorescence with complementary RNA. FIT-PNAs were conjugated to CLIP directly or via a PEG linker (where n=3, 8, or 12).

FIGS. 5A-B depict the added value of the PEG8 linker. Fluorescence spectra of CLIP6 FIT-PNA (5A, right) and, CLIP6-PEG8-FIT-PNA (5B, left) with fully matched RNA (mutant) and mis-match RNA (WT).

FIG. 6 depicts the increase in fluorescence for BRAF FIT-PNA substituted with cp. Fluorescence spectra of CLIP6-PEG8-cpFIT-PNA (1 μM) with fully matched RNA (mutant, (1.5 μM)) and mis-match RNA (WT, (1.5 μM)). A two-fold increase in fluorescence is obtained in comparison to the non-modified FIT-PNA (CLIP6-PEG8-FIT-PNA). An 18-fold enhancement in fluorescence is observed for CLIP6-PEG8-cpFIT-PNA in duplex form (with complementary RNA) in comparison to single strand.

FIG. 7 depicts an increase in fluorescence for CCAT1 FIT-PNA substituted with G⁺ as a neighboring base to BisQ at its 3′ position. A 7-fold increase in fluorescence is observed for G⁺ modified CCAT1 FIT-PNA in comparison to a ca. 3-fold increase for non-modified CCAT1 FIT-PNA ([RNA]=1.5 μM and [FIT-PNA]=1 μM).

FIG. 8 depicts the increase in fluorescence for CCAT1 FIT-PNA substituted with cpG⁺ as a neighboring base to BisQ at its 3′ position. A 10-fold increase in fluorescence is observed for cpG⁺ modified CCAT1 FIT-PNA; much higher than that observed for other modifications (i.e. G⁺ and cpG). ([RNA]=1.5 μM and [FIT-PNA]=1 μM).

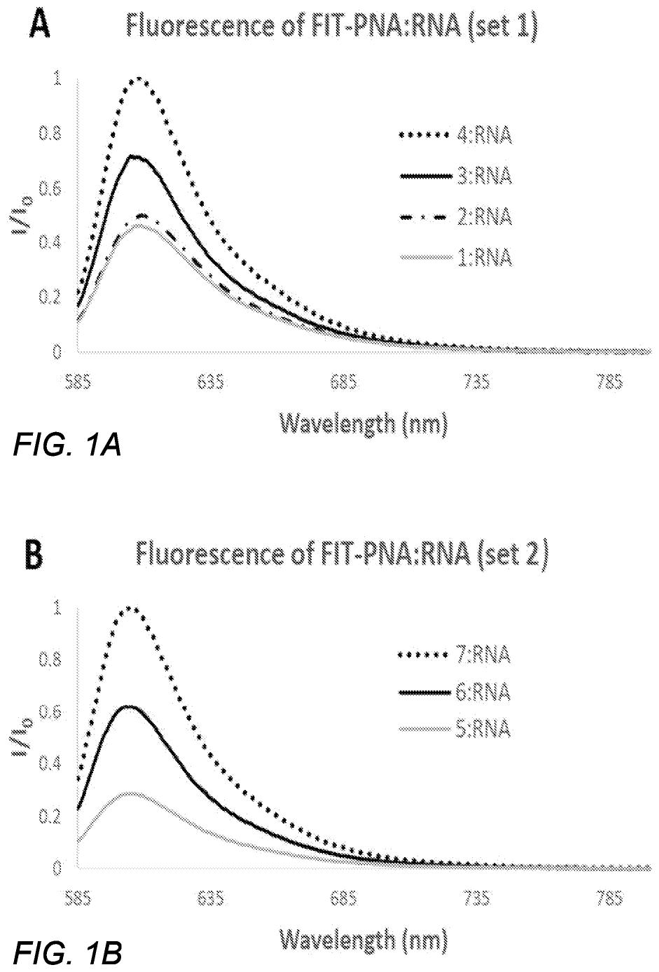

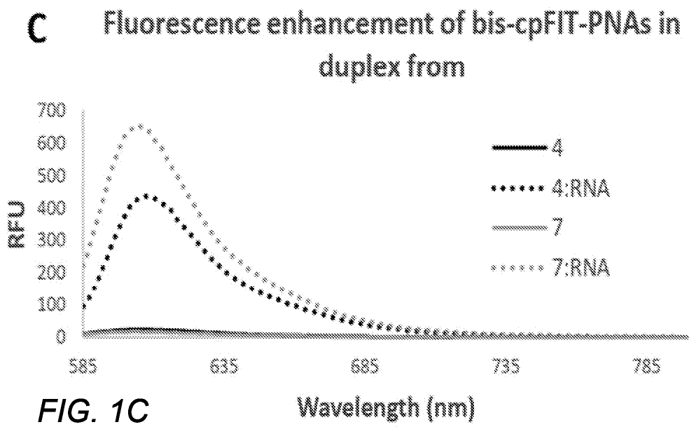

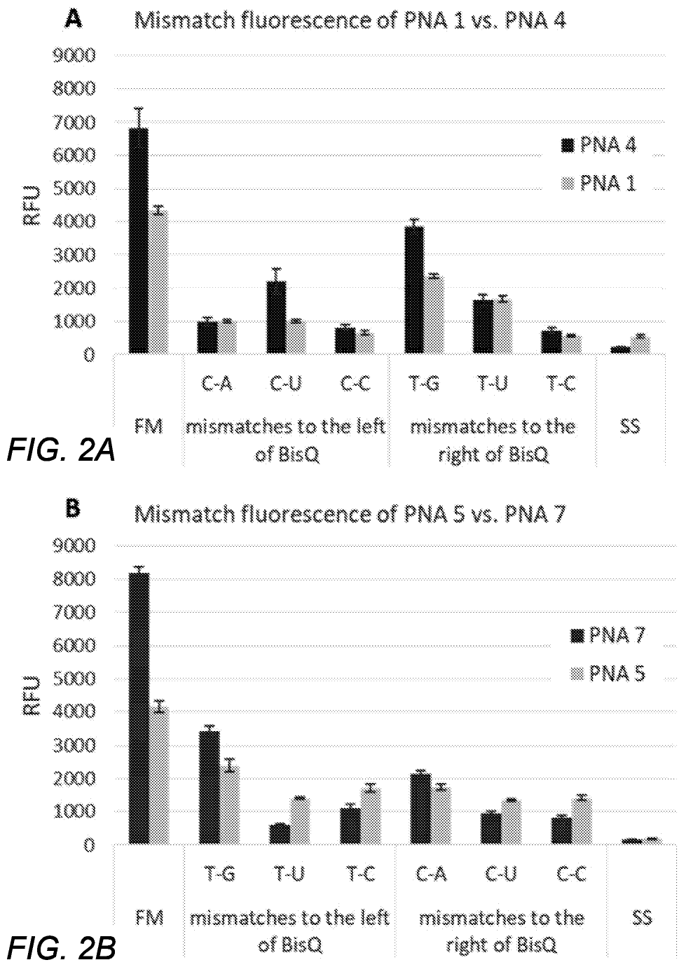

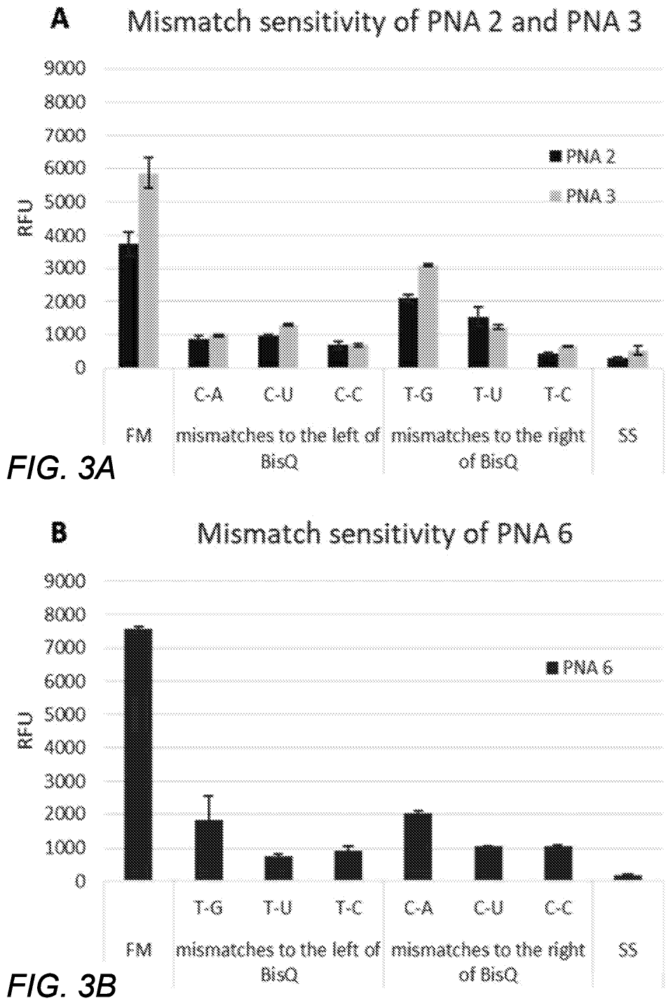

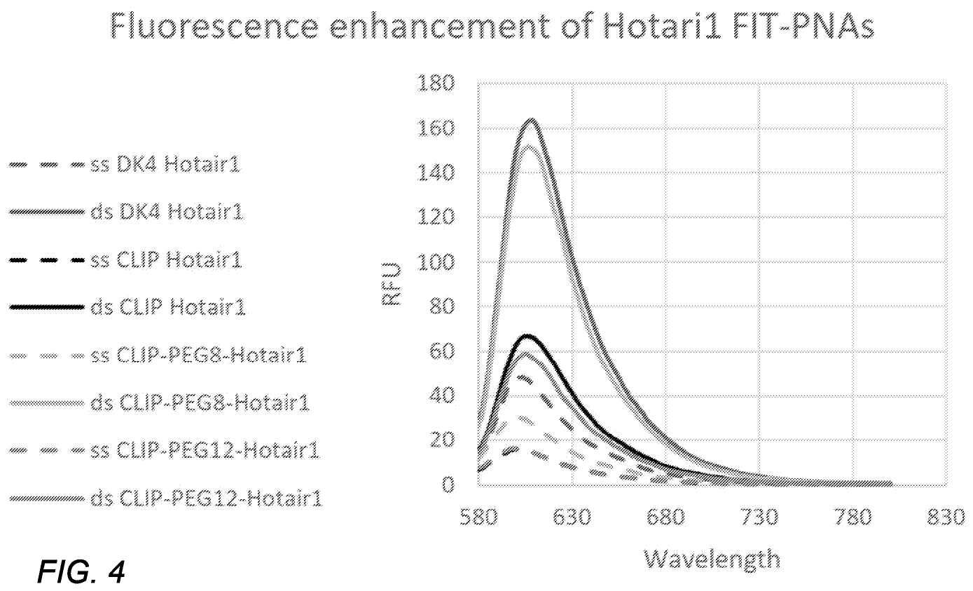

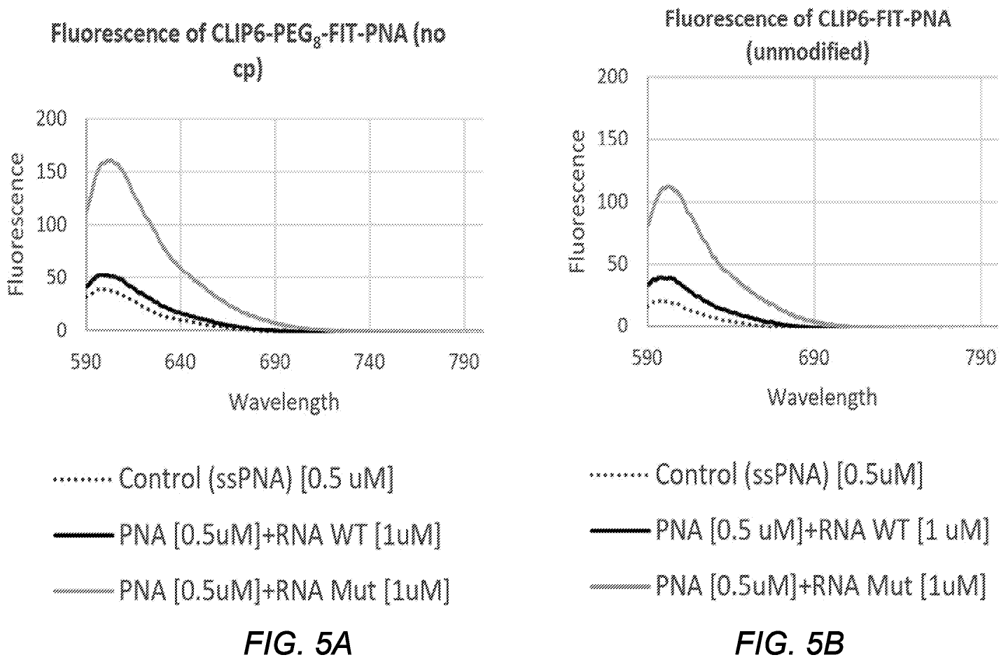

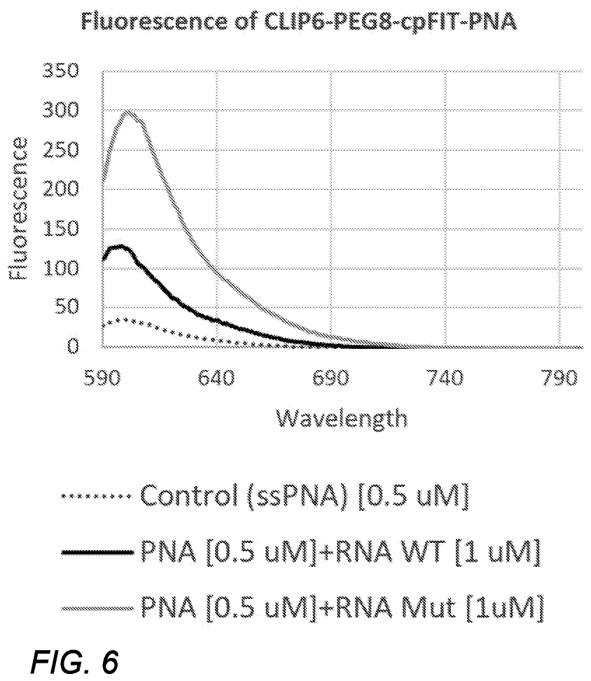

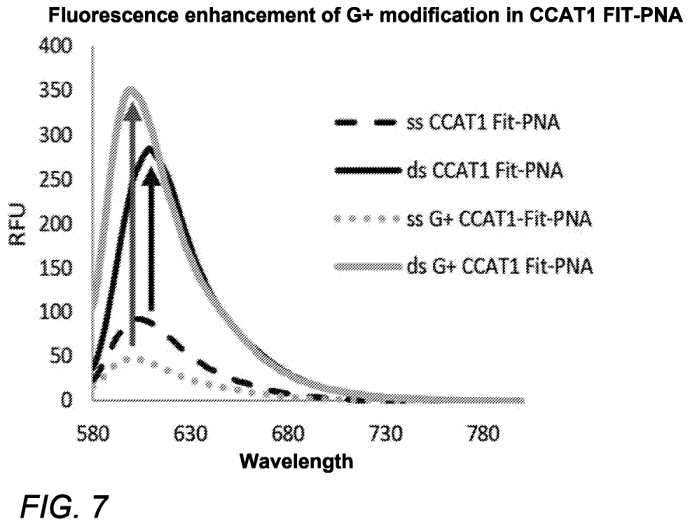

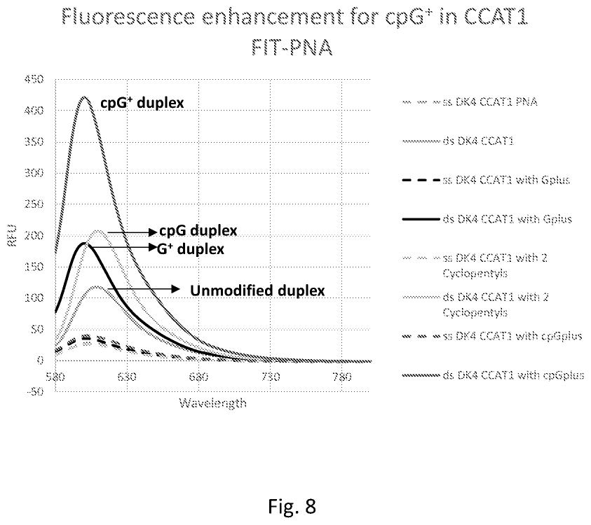

### DETAILED DESCRIPTION OF EMBODIMENTS

### General Procedures and Materials

Manual solid-phase synthesis was performed by using 5 mL polyethylene syringe reactors (Phenomenex) that are equipped with a fritted disk. All column chromatography was performed using 60A, 0.04-0.063 mm Silica gel (Biolab, Israel) and manual glass columns. TLC was performed using Merck Silica Gel 60 F254 plates. HPLC purifications and analysis were performed on a Shimadzu LC-1090 system using a semi-preparative C18 reversed-phase column (Jupiter C18, 5u, 300 Å, 250×10 mm, Phenomenex) at 50° C. Eluents: A (0.1% TFA in water) and B (MeCN) were used in a linear gradient (11-40% B in 38 min) with a flow rate of 4 mL/min. NMR spectra were recorded on a 300 and 600 MHz Bruker NMR using deuterated solvents as internal standards. Mass analysis of PNAs was acquired on a TSQ Quantum Access Max (Thermo Fisher Scientifc, Basel, Switzerland) mass spectrometer. The analysis was performed by direct injection into the mass spectrometer using electrospray ionization (ESI) in positive mode and full scan analysis (range of 200-1500 m/z).

RNA oligos were purchased from IDT, USA. Fmoc/Bhoc protected PNA monomers from PolyOrg Inc. (USA). Fmoc-D-Lysine and reagents for solid phase synthesis were purchased from Merck (Germany) and Biolab (Israel). Fmoc-protected cyclopentane PNA monomers (C and T) and BisQ were synthesized as previously reported.

Solid phase synthesis of cpFIT-PNA and FIT-PNA

Coupling of D-Lysine onto Novasyn TGA Resin. The resin (250 mg, 0.2 mmol/g) was allowed to swell in 10 ml DMF for 30 min. For pre-activation, DIC (5 eq.) and DIMAP (0.1 eq.) were added to a solution of Fmoc-protected D-Lysine (10 eq.) in DCM (15 ml) in an ice bath. After 15 min, the mixture was evaporated, re-dissolved in dry DMF and added to the resin. After 2.5 h, the resin was washed with DMF (5×2 mL), CH₂Cl₂ (5×2 mL) and the procedure was repeated.

Fmoc Cleavage. A solution of DMF/piperidine (4:1, 1 ml) was added to the resin. After 2 min the procedure was repeated. Finally, the resin was washed with DMF (3×1 ml), DCM (3×1 ml).

Coupling of Fmoc-Bhoc-PNA-Monomers. 4 eq. of PNA monomer, 4 eq. HATU, 4 eq. HOBt and 8 eq. of dry DIPEA in DMF (to 0.1 M PNA) were mixed in a glass vial equipped with a screw cap. After 3 min of pre-activation, the solution was transferred to the resin. After 60 min, the reaction mixture was discarded and the resin was washed with DMF (2×1 ml) and DCM (2×1 ml).

Coupling of BisQ. 4 eq. of BisQ monomer, 4 eq. HATU, 4 eq. HOBt and 8 eq. of dry DIPEA in DMF (to 0.1 M BisQ monomer) were mixed in a glass vial equipped with screw cap. Following 3 min of pre-activation, the solution was transferred to the resin. After 60 min, the procedure was repeated and finally the resin was washed with DMF (2×1 ml) and DCM (2×1 ml).

Coupling of cyclopentane modified PNAs. 4 eq. of cpPNA monomer, 4 eq. HATU, 4 eq. HOBt and 8 eq. of dry DIPEA in DMF (to 0.1 M cpPNA) were mixed in a glass vial equipped with screw cap. Following 3 min of pre-activation, the solution was transferred to the resin and shaked for 135 min. Finally, the resin was washed with DMF (2×1 ml) and DCM (2×1 ml).

Cleavage of PNA from resin. 1 ml TFA was added to the dry resin. After 2h another portion of TFA was added. The combined TFA solutions were concentrated in vacuo.

PNA Purification. PNAs were precipitated from the concentrated TFA solution by addition of cold diethyl ether (10 ml). The precipitate was collected by centrifugation and decantation of the supernatant. The residue was dissolved in water and purified by semi preparative HPLC. The purified PNAs were analysed by ESI-MS.

### Fluorescence Spectrometry

Fluorescence spectra were recorded by using a Jasco FT-6500 spectrometer. Measurements were carried out in fluorescence quartz cuvettes (10 mm) at a 3 μM concentration of FIT-PNA in a PBS buffered solution (100 mM NaCl, 10 mM NaH₂PO₄, pH 7). Quantum yields were determined relative to Cresyl Violet in PBS³. PNAs were hybridized to complementary RNA using a 1:1.5 mixture of PNA:RNA at 37° C. for 1-2 hr. Samples were excited at 575 nm and emission spectra were recorded at 585-800 nm.

Fluorescence end points were recorded by using a Cytation 3 plate reader. Measurements were carried out in Greiner 96 well black plates with flat bottom, at 0.5 μM concentration in a PBS buffered solution (100 mM NaCl, 10 mM NaH₂PO₄, pH 7). FIT-PNAs were hybridized to complementary RNA using a 1:1.5 mixture of PNA:RNA at 37° C. for 1-2 hr or by allowing overnight incubation at RT (for limit of detection (LOD) measurements). Samples were excited at 575 nm and measured at 615 nm.

### Synthesis

I— Exemplary synthesis of a hybrid cyclopentane-modified BisQ PNA monomer (cpBisQ) is shown in Scheme 6 (selective deprotection of tBOC is marked with a star).

The Synthesis is Based on:

H. Zheng et. al., Org. Lett. 2018, 20, 7637-7640; and

L. S. Lin et. al., Tetrahedron Letters 41 (2000) 7013-7016.

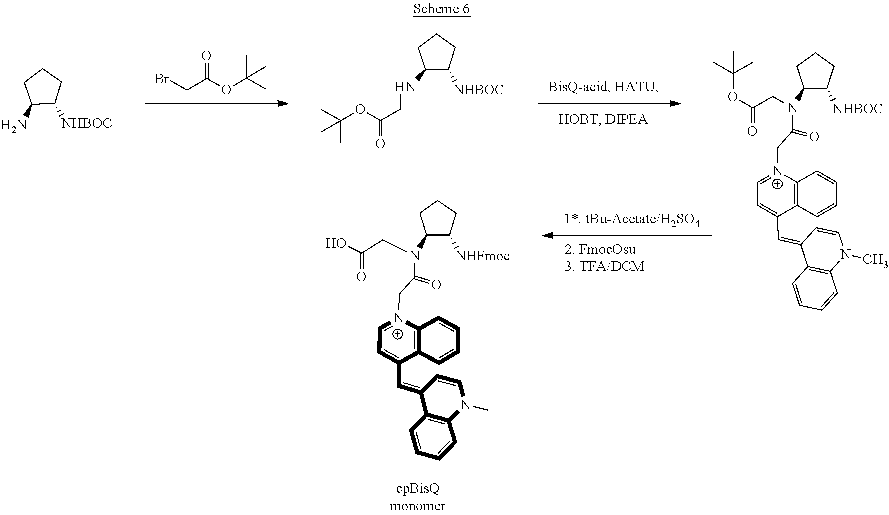

II— Exemplary synthesis of cpG+ is depicted in Scheme 7. The same approach may be implemented for the cpA monomer (cpA+).

The synthesis is based on M. Hibino et. al., Chem. Commun., 2020, 56, 25462549.

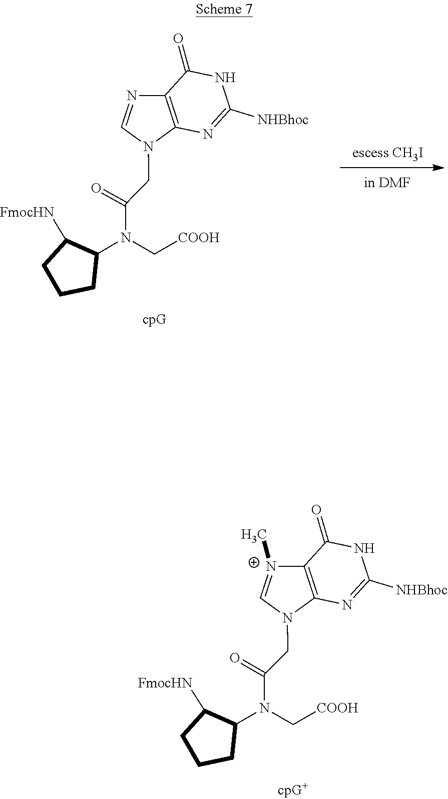

III— Exemplary synthesis of Ox-BisQ PNA monomer (Scheme 8).

The Synthesis is Based on:

S. Roesner et. al., Org. Biomol. Chem., 2020, 18, 5400-5405, and WO 2019/186174.

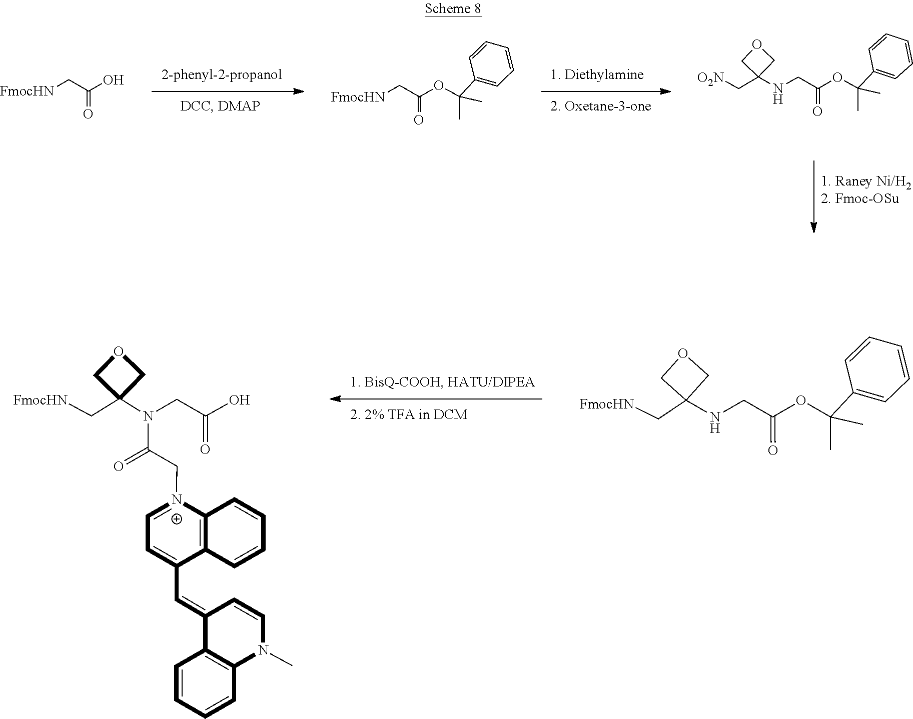

1-60. (canceled)

61. A cyclopentyl-modified PNA comprising a PNA backbone and a plurality of pendant nucleobases, at least one of said pendent bases is a surrogate base and wherein one or more cyclopentyl groups are provided in proximity to said surrogate base, wherein the surrogate base is a monomethine dye.

62. A cyclopentyl-modified forced-intercalation-peptide nucleic acid molecule (FIT-PNA) comprising a PNA backbone and a plurality of pendant nucleobases, at least one of said pendent bases is a surrogate base selected amongst monomethine dyes and wherein one or more cyclopentyl groups are provided in proximity to said surrogate base.

63. The PNA according to claim 61, wherein the monomethine dye is a cyanine dye being BisQ or a dye herein designated Dye 1 or Dye 2, wherein each X represents a halogen atom:

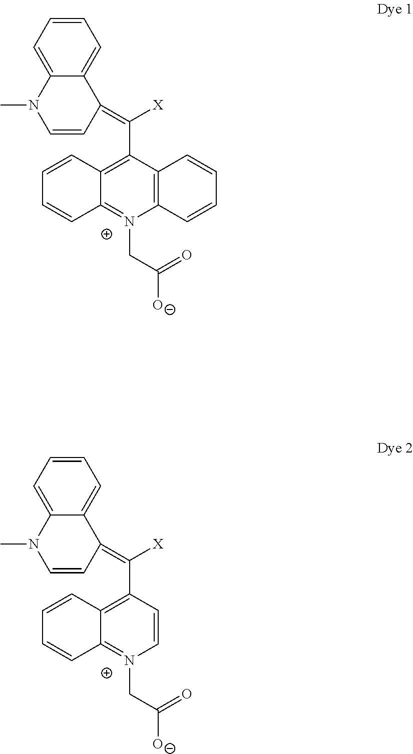

64. The PNA according to claim 61, wherein the one or more cyclopentyl (cp) group is positioned at a distance from the surrogate base that is not greater than one nucleobase.

65. The PNA according to claim 64, wherein the cp group is positioned on the PNA backbone between the position of the surrogate base and a position of the next nucleotide base, across a nucleotide base or on the PNA unit carrying the surrogate base.

66. The PNA according to claim 64, wherein the cp group is on the 3′ end of the PNA or on the 5′ end of the PNA.

67. The PNA according to claim 61, further comprising a charged guanine (G+) and/or a charged adenine (A+) nucleobase.

68. The PNA according to claim 67, wherein the charged G+ and/or the charged A+ is an alkylation product of a free nucleobase G and/or A.

69. The PNA according to claim 67, wherein the charged nucleobases having the structures: 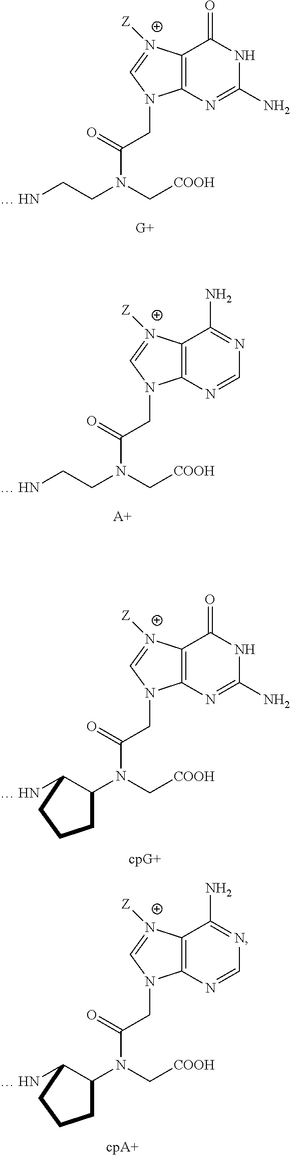
wherein each of Z, independently, is an alkyl having between 1 and 5 carbon atoms.

70. The PNA according to claim 61, wherein the PNA further comprises an oxetane-modified PNA unit of the structure

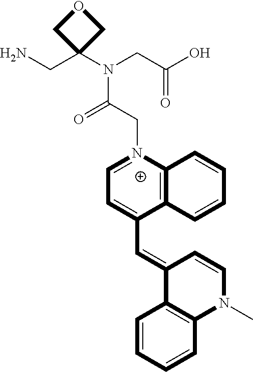

71. The PNA according claim 61, the PNA comprising:
(i) cp-modified PNA unit, said unit optionally being the surrogate base bearing unit; or
(ii) cp-modified PNA unit, said unit being a BisQ-bearing unit; or
(iii) cp-modified PNA unit, said unit being optionally a G+ or A+ nucelobase; or
(iv) cp-modified PNA unit, said unit being a BisQ-bearing unit; the PNA further comprising a G+ or A+ nucleobase, being optionally cpG+ and cpA+ nucleobase; or
(v) cp-modified PNA unit, said unit being a BisQ-bearing unit; the PNA further comprising an oxetane-modified PNA base; or
(vi) cp-modified PNA unit, said unit being a BisQ-bearing unit; the PNA further comprising a G+ and/or A+ base, a cpG+ and/or cpA+ base, and/or an oxetane-modified PNA base.

72. A method for detecting or for determining presence of a sequence of interest (SOI) in a sample, the method comprising contacting said sample with a PNA according to claim 61, under conditions permitting hybridization of said PNA with the SOI and detecting emission of light in the red-to-NIR region upon exposure to red-to-NIR radiation.

73. The method according to claim 72, wherein said contacting comprises incubating the PNA and the sample for a period of time to allow for hybridization of the PNA with the SOI.

74. The method according to claim 72, wherein the sample is in vivo or ex vivo.

75. The method according to claim 72, for determining malignancies in a fluorescence guided surgery.

76. The method according to claim 72, wherein the SOI is an RNA or a DNA sequence associated with a disease or a condition, indicative of a genetic condition or a pathogenic RNA/DNA.

77. The method according to claim 76, wherein the SOI is an oncogenic RNA or a pathogenic RNA.

78. The method according to claim 77, wherein the SOI is a DNA.

79. The method according to claim 72, for detecting a single point mutation associated with a genetic disorder.

80. A method for determining presence, development or progression of a disease or a condition; or a genetic condition; or presence of a pathogen or a parasite in a sample, the method comprising contacting a sample containing or suspected of containing a sequence of interest (SOI) indicative of presence of a disease, condition or pathogen or parasite in a subject with a PNA according to claim 61 under conditions permitting hybridization of said PNA with the SOI and detecting emission of light in the red-to-NIR region upon exposure to red-to-NIR radiation.
